(19) United States
(12) Patent Application Publication
(10) Pub. No.: US 2019/0374613 A1
(43) Pub. Date: Dec. 12, 2019

(54) PHARMACEUTICAL COMPOSITIONS OF GLP-1 ANALOGUES

(71) Applicant: Biocon Research Limited, Karnataka (IN)

(72) Inventors: Parikshit KHOKALE, Karnataka (IN); Narayanan KASINATHAN, Electronic City Post (IN)

(21) Appl. No.: 16/463,086

(22) PCT Filed: Nov. 22, 2017
(86) PCT No.: PCT/IB2017/057321

§ 371 Date: May 22, 2019

(30) Foreign Application Priority Data
Nov. 22, 2016 (IN) 201641039908

(51) Int. Cl. A61K 38/26; A61K 9/00

(52) U.S. Cl. CPC A61K 38/26; A61K 9/0019

(57) ABSTRACT

The present invention relates to pharmaceutical composition comprising: (a) Liraglutide as active drug substance; (b) Buffer selected from the group consisting of Sodium citrate, arginine, disodium hydrogen phosphate, glycine or any combination thereof; (c) tonicity modifier selected from glycerol, mannitol, propylene glycol, xylitol and trehalose and (d) preservative selected from phenol, cresol, resorcinol, methyl paraben, propyl paraben or any combination thereof. This invention also relates to processes for the preparation of said pharmaceutical compositions.

### RELATED APPLICATION

This application claims the benefit of priority of our Indian patent applications IN 201641039908 filed on Nov. 22, 2016 which is incorporated herein by reference.

### TECHNICAL FIELD

Liraglutide is an analogue of GLP-1 and it acts as GLP-1 receptor agonist. It has 97% amino acid homology to human GLP-1 (AA 7 to 37). GLP-1 has been engineered at position 34, where lysine is replaced by Arginine.

Liraglutide is an injectable drug used for reducing the level of sugar (glucose) in the blood. It is used for treating type 2 diabetes.

Liraglutide belongs to a class of drugs called incretin mimetics because, these drugs mimic the effects of incretins. Incretins, such as human-glucagon-like peptide-1 (GLP-1), are hormones that are produced and released into the blood by the intestine in response to food.

GLP-1 increases the secretion of insulin from the pancreas, slows absorption of glucose from the gut, and reduces the action of glucagon. (Glucagon is a hormone that increases glucose production by the liver.)

All three of these actions reduce levels of glucose in the blood. In addition, GLP-1 reduces appetite. Liraglutide is a synthetic GLP- 1 analogue.

### BACKGROUND AND PRIOR ART OF THE DISCLOSURE

Victoza® (liraglutide [rDNA origin] injection), solution for subcutaneous use got initial U.S. Approval in 2010. Victoza is a glucagon-like peptide-1 (GLP-1) receptor agonist indicated as an adjunct to diet and exercise to improve glycemic control in adults with type 2 diabetes mellitus (1). Victoza® is a registered trademark of Novo Nordisk A/S.

U.S. Pat. No. 6,268,343 discloses Liraglutide and its formulations.

A few efforts are made in the prior art to provide suitable compositions of liraglutide.

U.S. Pat. Nos. 8,846,618 and 8,114,833 discloses the composition of the liraglutide injectable formulation in which a) Liraglutide is the active ingredient b) Propylene glycol is tonicity modifier c) disodium hydrogen phosphate is used as buffering agent, Phenol as preservative and WFI as vehicle. The Propylene glycol present in said formulation at concentration about 4 to 50 mg/ml and formulation has pH from 7.0 to 10.0.

WO 2016/038521 A1 discloses the composition of the liraglutide injectable formulation with dipotassium phosphate, sodium bicarbonate and Disodium hydrogen phosphate anhydrous as buffers.

CN105126082 A discloses liraglutide drug preparation with a mixture of citric acid and carbonate as a buffer salt system. The preparation comprises liraglutide, a buffer agent, an isotonic adjusting agent or a stabilizer and a preservative.

WO 2017147783 discloses liraglutide compositions and processes for their preparation.

There is a need to develop stable alternative formulations of liraglutide which are easy to manufacture. The present invention addresses the above need with stable and easy to manufacture formulations.

### SUMMARY OF THE INVENTION

The present invention relates to pharmaceutical compositions comprising liraglutide as active drug substance, buffers from group of Sodium citrate, arginine, Disodium hydrogen phosphate and glycine, tonicity modifier from glycerol, mannitol, propylene glycol, xylitol and trehalose along with a preservative.

Stability of these pharmaceutical composition is found to be improved under stressed conditions. Impurities such as higher molecular weight variants, hydrophilic variants and total impurities are found to be controlled. Rate of impurity generation is significantly low as compared with the prior arts.

### DETAILED DESCRIPTION OF THE INVENTION

An aspect of the present invention is to provide a pharmaceutical composition comprising liraglutide as active drug substance, buffers from group of Sodium citrate, arginine, disodium hydrogen phosphate and glycine, tonicity modifier from glycerol, mannitol, propylene glycol, xylitol and trehalose along with a preservative.

A pharmaceutical composition comprising:
a) Liraglutide as active drug substance.
b) Buffer selected from the group consisting of Sodium citrate, arginine, disodium hydrogen phosphate, glycine or any combination thereof.
c) A tonicity modifier selected from glycerol, mannitol, propylene glycol, xylitol and trehalose.
d) A preservative selected from phenol, cresol, resorcinol, methyl paraben, propyl paraben or any combination thereof.

According to one embodiment of this aspect, the preservative is selected from the group consisting of phenol, cresols, resorcinol, methyl paraben, propyl paraben and the like.

Another aspect of the present invention provides a process for the preparation of the pharmaceutical composition of liraglutide comprising:

(i) dissolving in water a buffer selected from the group consisting of glycine, arginine, sodium citrate and disodium hydrogen phosphate; a tonicity modifier from glycerol, mannitol, propylene glycol, xylitol and trehalose; and a preservative;

(ii) adding liraglutide to the solution of step (i) and stirring to dissolve;

(iii) adjusting the pH of the solution of step (ii) with a pH-adjusting agent to a pH range from about 7.0 to about 8.5.

According to one embodiment of this aspect, the preservative is selected from the group consisting of phenol, cresols, resorcinol, methyl paraben, propyl paraben and the like.

The term “liraglutide,” as used herein, includes liraglutide as well as its pharmaceutically acceptable salts, hydrates, solvates, prodrugs, chelates, and complexes.

The term “buffer,” as used herein, refers to a chemical compound added to a liraglutide composition to prevent the pH from changing with time. Suitable buffers are selected from the group consisting of Sodium citrate, arginine, disodium hydrogen phosphate and glycine.

Propylene glycol is preferably used as an isotonic agent in the pharmaceutical compositions of the present invention. However, it can be replaced or used in combination with glycerol, mannitol, propylene glycol, xylitol and trehalose or combinations thereof.

Suitable preservatives are selected from the group consisting of phenol, cresol, resorcinol, methyl paraben, propyl paraben and the like.

The term “pH-adjusting agent,” as used herein, refers to an agent used to adjust the pH in the desired range. Suitable examples of pH-adjusting agents include sodium hydroxide, potassium hydroxide and hydrochloric acid.

### BRIEF DESCRIPTION OF THE FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

FIG. 1 Illustrates the solubility profile of the Liraglutide Drug substance (DS) at different pH.

FIG. 2 Illustrates total impurities trends observed for different buffers at stressed condition.

FIG. 3 Illustrates degradation rate (i.e. level of Total impurities) observed for different tonicity modifier.

FIG. 4 Illustrates comparison of degradation rates (i.e. level of total impurities) observed in selected formulations.

Liraglutide has pI around 4.9 and its dissolution in the aqueous vehicle can be facilitated by selection of optimum pH. Based on the prior art only higher pH than pI is screened for the dissolution and stability.

pH screening was performed with 12 mg/ml of GLP-1 analogue were 6.0, 6.5, 7.0, 7.5, 8.0, 8.5, and 9.0. At fixed volume and agitation the solubility was checked in buffer maintained at different pH as mentioned above. Solubility was checked by visual analysis and analysis of solution by HPLC based quantitation.

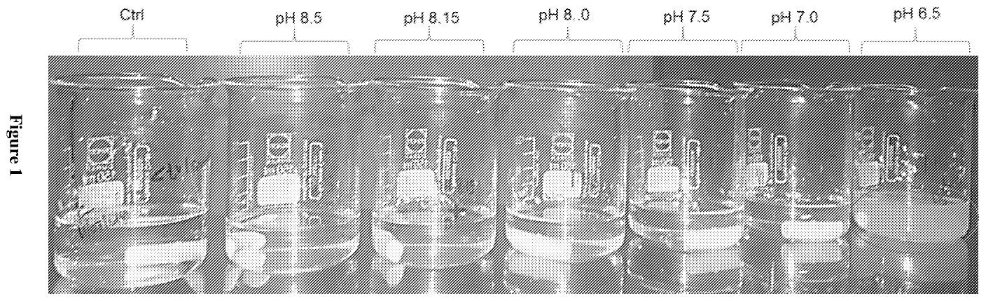

Solubility profile of the Liraglutide Drug substance (DS) at different pH is illustrated in FIG. 1.

Below pH 7.0, DS was not completely soluble even after 30 min of stirring (FIG. 1). Liraglutide was completely soluble at pH 7.5 and above.

TABLE 1
Solubility analysis
Solubility analysts - at 12 mg/ml

pH

| | 6.0 | 6.5 | 7 | 7.5 | 8 | 8.5 | 9 | 9.5 |
|---|---|---|---|---|---|---|---|---|
| Assay (%) | 1 | 14 | 92 | 90 | 90 | 94 | 90 | 96 |

As found from the visual analysis and confirmed by content analysis (Table 1) by HPLC based method, it was observed that above pH 7.0, DS shows maximum solubility.

To avoid generation of impurities at higher alkaline condition, exposure of higher pH was avoided. Liraglutide was dissolved at near neutral pH, after complete dissolution pH was adjusted to target pH. Dissolution was carried out at pH 6.4-7.5, after complete dissolution pH of the solution adjusted to 7.0-8.5.

Buffer for Liraglutide Formulation:

Buffers used in the preparation of formulation of Liraglutide were Arginine (L), Glutamic acid (Monosodium- monohydrate), Glycine, Histidine, Disodium hydrogen phosphate dihydrate, Sodium phosphate monobasic monohydrate, Sodium citrate, Sodium bicarbonate and TRIS (Table-2). The drug substance was mixed with each buffers and analyzed for solubility and degradation kinetics.

Concentration of these buffer was from 3 mM to 100 mM.

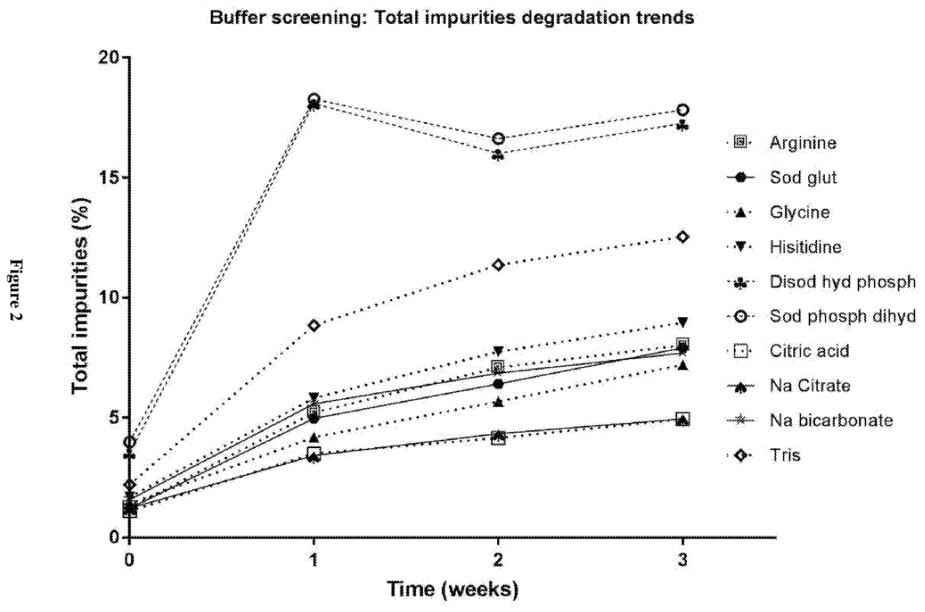

Based on the stability profiles at stressed conditions (FIG. 2), and rate of rise in total impurities, sodium citrate, arginine, glycine and disodium hydrogen phosphate were selected from the group.

Concentration range for the optimal buffers was but not limited to 3 to 60 mM.

TABLE 2
Kinetics of degradation with respect to total impurities observed
with different buffers at stressed conditions

| | | | | | Rate of |
|---|---|---|---|---|---|
| | | | | | degradation |
| | Time (Weeks) | | | | (Inpurity rise |
| Buffer + DS | 0 | 1 | 2 | 3 | (Δ)/week) |
| Arginine + DS | 1.25 | 5.21 | 7.08 | 8.02 | 2.22 |
| Sodium glutamate + | 1.22 | 4.96 | 6.4 | 7.9 | 2.15 |
| DS | | | | | |
| Glycine + DS | 1.41 | 4.18 | 5.67 | 7.2 | 1.89 |
| Hisitidine + DS | 1.69 | 5.81 | 7.74 | 8.95 | 2.37 |
| Disodium hydrogen | 3.54 | 18.09 | 16.01 | 17.26 | 3.91 |
| phosphate + DS | | | | | |
| Sodium phosphate | 3.98 | 18.27 | 16.63 | 17.82 | 3.99 |
| dihydrogen + DS | | | | | |
| Citric acid + DS | 1.1 | 3.5 | 4.15 | 4.94 | 1.22 |
| Na Citrate + DS | 1.22 | 3.42 | 4.33 | 4.95 | 1.21 |
| Na bicarbonate + DS | 1.58 | 5.57 | 6.85 | 7.69 | 1.96 |
| Tris + DS | 2.2 | 8.84 | 11.36 | 12.53 | 3.35 |

Total impurities trends observed for different buffers at stressed condition are ollustrated in FIG. 2.

Screening of Tonicity Modifiers for Liraglutide Formulation

Different tonicity modifiers were screened with liraglutide at different concentrations. Tonicity was measured in terms of mOsmol per Kg. List of tonicity modifiers studied were Arginine (L), Glycerol, Histidine, Mannitol, Propylene glycol, Sodium chloride, Sorbitol, Sucrose, Trehalose and Xylitol. The concentration of the tonicity modifiers was maintained to achieve osmolality as per Table 3.

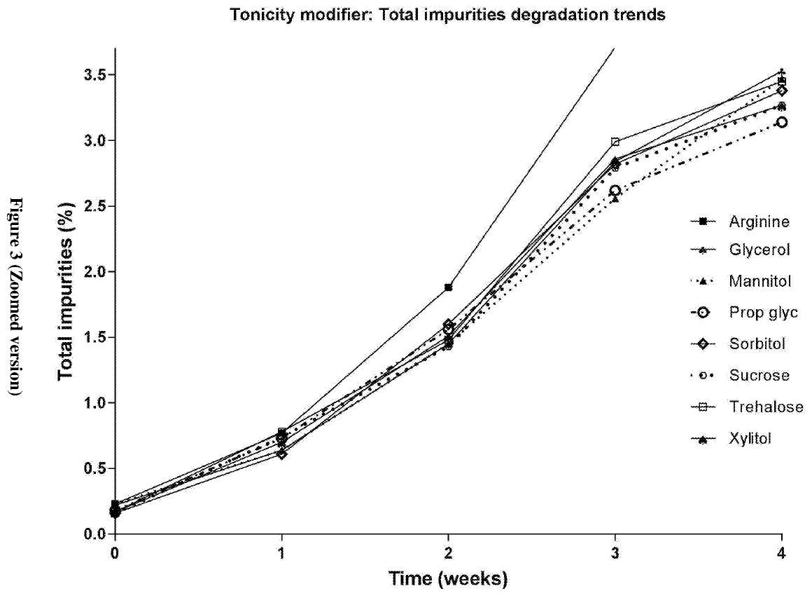

Based on degradation rate with respect to total impurities (FIG. 3), glycerol, mannitol, propylene glycol, trehalose and xylitol were selected as tonicity modifiers.

TABLE 3
Tonicity modifier concentration

| | Osmolality (mOsmol/ |
|---|---|
| Tonicity modifier | Kg) |
| Arginine (L) | 273 |
| Glycerol | 250 |
| Histidine | 663 |
| Mannitol | 285 |
| Propylene glycol | 240 |
| NaCl | 345 |
| Sorbitol | 304 |
| Sucrose | 353 |
| Trehalose | 365 |
| Xylitol | 338 |

Degradation rate (i.e. level of Total impurities) observed for different tonicity modifier is illustrated in FIG. 3.

TABLE 4
Kinetics of degradation with respect to total impurities observed
with different tonicity modifiers at stressed conditions
Total impurities (%)

| | | | | | | Rate of |
|---|---|---|---|---|---|---|
| | | | | | | degradation |
| Tonicity | Time (Weeks) | | | | | (Impurity rise |
| modifier | 0 | 1 | 2 | 3 | 4 | (Δ)/week) |
| Arginine | 0.23 | 0.77 | 1.88 | 3.71 | 4.63 | 1.17 |
| Glycerol | 0.22 | 0.64 | 1.45 | 2.84 | 3.53 | 0.88 |
| Mannitol | 0.23 | 0.64 | 1.45 | 2.56 | 3.47 | 0.84 |
| Propylene | 0.18 | 0.73 | 1.56 | 2.62 | 3.14 | 0.78 |
| glycol | | | | | | |
| Sorbitol | 0.16 | 0.61 | 1.6 | 2.82 | 3.38 | 0.87 |
| Sucrose | 0.16 | 0.74 | 1.43 | 2.79 | 3.27 | 0.83 |
| Trehalose | 0.17 | 0.78 | 1.48 | 2.99 | 3.45 | 0.88 |
| Xylitol | 0.17 | 0.7 | 1.51 | 2.86 | 3.27 | 0.84 |

The concentration of the above studied tonicity modifiers were but not limited to a range as mentioned below.

The concentration range of Glycerol was from 0.5 to 5%.

The concentration range of mannitol was from 1 to 20%.

The concentration range of propylene glycol was from 0.5 to 10%.

The concentration range of trehalose was from 0.2 to 20%.

The concentration range of xylitol was from 0.5 to 50%.

Manufacturing Process of the Liraglutide Formulation:

Method of preparing liraglutide formulation comprises of following steps:

1. Dissolution of preservative, buffer and tonicity modifier into sufficient water for injection.

2. Add drug substance to the above solution, mix it to dissolve.

3. Adjust pH by adding NaOH/HCl.

4. pH of the final solution should be in between 7.0-8.5.

Optimal Formulation Composition:

Formulations were prepared from screened buffers and tonicity modifiers were prepared and analysed for total impurities, high molecular weight variants and hydrophilic impurities. Rate of degradation (i.e rate of rise in each of the impurity) at stressed conditions were compared. Different compositions of selected buffers and selected tonicity modifier prepared using preservative and Liraglutide are given in table 5.

TABLE 5
Examples of Screened formulations

| Formulation | | | | |
|---|---|---|---|---|
| | Tonicity | | GLP-1 | Formulation |
| Buffer | modifier | Preservative | analogue | code |
| Arginine | Glycerol (G) | Phenol | Liraglutide | Ar + G |
| (Ar) | Mannitol (M) | | | Ar + M |
| | Trehalose (T) | | | Ar + T |
| | Xylitol (X) | | | Ar + X |
| | Propylene | | | Ar + P |
| | glycol (P) | | | |
| Glycine | Glycerol (G) | | | Gl + G |
| (Gl) | Mannitol (M) | | | Gl M |
| | Trehalose (T) | | | Gl + T |
| | Xylitol (X) | | | Gl + X |
| | Propylene | | | Gl + P |
| | glycol (P) | | | |
| Sodium | Glycerol (G) | | | Cit + G |
| citrate | Mannitol (M) | | | Cit + M |
| (Cit) | Trehalose (T) | | | Cit + T |
| | Xylitol (X) | | | Cit + X |
| | Propylene | | | Cit + P |
| | glycol (P) | | | |
| Disodium | Glycerol (G) | | | Pho + G |
| hydrogen | Mannitol (M) | | | Pho + M |
| phosphate | Trehalose (T) | | | Pho + T |
| (Pho) | Xylitol (X) | | | Pho + X |
| | Propylene | | | Pho + P |
| | glycol (P) | | | |

The concentration of buffers (i.e Arginine, Glycine, Sodium citrate and Disodium hydrogen phosphate) in the formulations was in the range but not limited to about 4 mM to 50 mM. The concentration of Tonicity modifiers were adjusted to maintain the osmolality.

The concentration range of Glycerol was about 1.2% to about 3.5%, concentration range of mannitol was about 1.5% to about 10%, concentration range of propylene glycol was about 0.5% to about 4%, concentration range of trehalose was about 0.2% to about 5%, and concentration range of xylitol was about 0.5% to about 30%.

Phenol was added to each formulation at about 3.5 to 6.0 mg/ml as final concentration as preservative and WFI used as vehicle.

The process for the preparation of Liraglutide pharmaceutical composition comprising:
a) dissolving a buffer selected from table 5 or in combination thereof; a tonicity modifier selected from table 5; and a preservative; in water
(b) adding liraglutide to the solution of step (a), mixing it to dissolve.
(c) adjusting pH of the solution of step (b) to a pH of from about 7.0-8.5.

Level of impurities were analysed immediately after preparation of formulation and across the stability time points. At each time point the level of impurities were quantitated and trends are plotted.

Total impurities, higher molecular weight variants and hydrophilic variants are analysed using reverse phase HPLC, high performance size exclusion chromatography and reverse phase HPLC methods respectively.

TABLE 6
Total impurities in formulations

| | Total impurities (%) | | | | | |
|---|---|---|---|---|---|---|
| | Time points (weeks) | | | | | |
| | 0 | 1 | 2 | 3 | 4 | Degradation rate |
| Ar + G | 0.4 | 1.0 | 1.9 | 3.0 | 4.1 | 0.94 |
| Ar + M | 0.2 | 1.1 | 1.8 | 2.8 | 3.9 | 0.91 |
| Ar + T | 0.3 | 1.0 | 1.8 | 2.8 | 3.9 | 0.90 |
| Ar + X | 0.2 | 1.1 | 1.9 | 2.9 | 3.9 | 0.91 |
| Ar + P | 0.2 | 1.0 | 1.8 | 2.9 | 4.0 | 0.94 |
| Gl + G | 0.2 | 0.9 | 1.6 | 2.6 | 4.0 | 0.93 |
| Gl M | 0.3 | 0.9 | 1.7 | 2.9 | 4.1 | 0.95 |
| Gl + T | 0.3 | 1.0 | 1.6 | 2.7 | 3.9 | 0.89 |
| Gl + X | 0.3 | 1.0 | 1.7 | 2.6 | 4.0 | 0.92 |
| Gl + P | 0.2 | 1.0 | 1.7 | 2.7 | 4.0 | 0.91 |
| Cit + G | 0.2 | 0.6 | 1.3 | 2.1 | 3.2 | 0.75 |
| Cit + M | 0.2 | 0.7 | 1.3 | 2.1 | 3.2 | 0.74 |
| Cit + T | 0.3 | 0.7 | 1.3 | 2.1 | 3.2 | 0.73 |
| Cit + X | 0.3 | 0.7 | 1.3 | 2.2 | 3.2 | 0.74 |
| Cit + P | 0.3 | 0.8 | 1.2 | 2.2 | 3.2 | 0.73 |
| Pho + G | 0.3 | 0.8 | 1.3 | 2.3 | 3.5 | 0.78 |
| Pho + M | 0.2 | 0.8 | 1.4 | 2.3 | 3.5 | 0.81 |
| Pho + T | 0.3 | 0.8 | 1.2 | 2.2 | 3.3 | 0.76 |
| Pho + X | 0.2 | 0.8 | 1.3 | 2.3 | 3.6 | 0.81 |
| Pho + P | 0.4 | 0.9 | 1.4 | 2.3 | 3.7 | 0.81 |
| Victoza lot 1 | 1.4 | 2.0 | 3.4 | 3.8 | 4.9 | 0.87 |
| Victoza lot 2 | 1.3 | 2.0 | 3.3 | 3.9 | 4.7 | 0.87 |
| Victoza lot 3 | 1.4 | 2.0 | 3.3 | 3.9 | 4.6 | 0.83 |
| Victoza lot 4 | 1.3 | 1.9 | 3.5 | 4.1 | 4.8 | 0.91 |
| Victoza lot 5 | 1.7 | 2.3 | 3.7 | 4.7 | 5.3 | 0.97 |

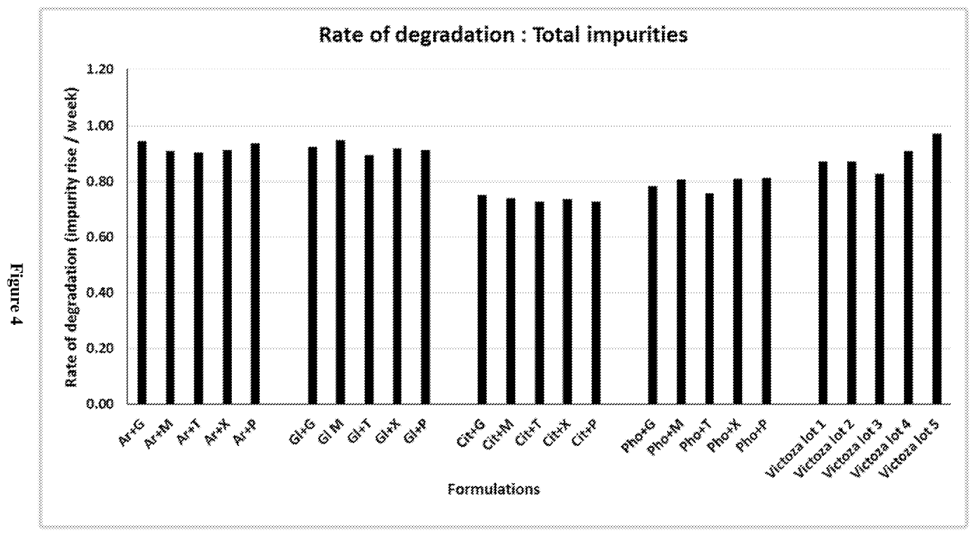

Comparison of degradation rates (i.e. level of total impurities) observed in selected formulation is illustrated in FIG. 4.

The above results demonstrate that the compositions as disclosed in the above table were stable.

1. A pharmaceutical composition comprising:
a) Liraglutide as active drug substance;
b) buffer selected from the group consisting of Sodium citrate, arginine, disodium hydrogen phosphate, glycine or any combination thereof;
c) a tonicity modifier selected from glycerol, mannitol, propylene glycol, xylitol and trehalose;
d) a preservative selected from phenol, cresol, resorcinol, methyl paraben, propyl paraben or any combination thereof.

2. The pharmaceutical composition of claim 1, wherein buffer is disodium hydrogen phosphate.

3. The pharmaceutical composition of claim 1, wherein buffer is sodium citrate.

4. The pharmaceutical composition of claim 1, wherein buffer is arginine.

5. The pharmaceutical composition of claim 1, wherein buffer is glycine.

6. The pharmaceutical composition of claim 1, wherein tonicity modifier is propylene glycol.

7. The pharmaceutical composition of claim 1, wherein tonicity modifier is glycerol.

8. The pharmaceutical composition of claim 1, wherein tonicity modifier is mannitol.

9. The pharmaceutical composition of claim 1, wherein tonicity modifier is selected from trehalose.

10. The pharmaceutical composition of claim 1, wherein tonicity modifier is xylitol.

11. The pharmaceutical composition according to claim 1, characterized in that, pH is maintained from 7.0-8.5.

12. A process for the preparation of the pharmaceutical composition of claim 1, wherein the process comprises:
a) dissolving a buffer selected from the group consisting of Sodium citrate, arginine, disodium hydrogen phosphate, glycine or any combination thereof; a tonicity modifier selected from propylene glycol, glycerol, mannitol, trehalose and mannitol; and a preservative; in water
(b) adding liraglutide to the solution of step (a), mixing it to dissolve.
(c) adjusting pH of the solution of step (b) to a pH of from about 7.0-8.5.
